(19) United States
(12) Patent Application Publication
(10) Pub. No.: US 2024/0273734 A1
(43) Pub. Date: Aug. 15, 2024

(54) MARKER AND MARKERLESS TRACKING

(71) Applicant: Northern Digital Inc., Waterloo (CA)

(72) Inventors: Larry Chen, Fergus (CA); Uditha Jayarathne, Waterloo (CA); Steven van Hengstum, Radolfzell (DE)

(21) Appl. No.: 18/438,018

(22) Filed: Feb. 9, 2024

Related U.S. Application Data
(60) Provisional application No. 63/484,625, filed on Feb. 13, 2023.

(51) Int. Cl. G06T 7/246; G06T 17/00; G06V 10/74; H04N 5/74

(52) U.S. Cl. CPC G06T 7/246; G06T 17/00; G06V 10/761; H04N 5/74; G06V 2201/07

(57) ABSTRACT

A system includes a projector to project a pattern of dots within a tracking volume, a medical instrument having markers and positioned within the tracking volume, an image capture unit to capture images of the medical instrument and the markers. The image capture unit captures images of the pattern of dots within the tracking volume, and a computing device performs operations that include initiating capture of at least two images of the medical instrument and the markers, determining a three-dimensional position of the markers from the captured images of the markers, initiating projection of the pattern of dots within the tracking volume, initiating capture, of at least two images of a portion of the pattern of dots, and determining three-dimensional positions of dots in the portion of dots from the captured images.

### CROSS-REFERENCE TO RELATED APPLICATIONS

This application claims the benefit of U.S. Provisional Patent Application No. 63/484,625 filed Feb. 13, 2023, which is incorporated herein by reference in its entirety.

### TECHNICAL FIELD

This disclosure relates to tracking objects by employing marker and markerless techniques.

### BACKGROUND

Tracking systems (e.g., optical tracking systems) used to track various types of objects (e.g., surgical tools, etc.) often rely on one or multiple markers (detectable by the system) being affixed to the objects. Such markers may be active markers (e.g., light emitting diode markers), passive markers, or a combination of active and passive markers. In some instances, passive markers can reflect an optical signal toward a camera (of the tracking system) that captures the reflected signal and provides data (representing the signal) to other components of the tracking system. From the provided data, the tracking system can estimate the position of the marker and track the object (that the maker is affixed) within an environment.

### SUMMARY

The described systems and methods use a single image capture unit to capture imagery associated with marker and markerless tracking. For example, the single image capture unit captures images of a surgical tool having one or more light-reflective markers and the same image capture unit also captures images of dots projected onto a patient (e.g., a portion of a patient such as the patient's face). From the captured imagery, position information, orientation information, etc. of the surgical tool can be attained along with anatomy information, orientation information, position information, etc. of the patient.

Advantageously, by employing a single image capture unit, the location of one capture unit (rather than multiple units) needs to be registered with the system. Further, only a single capture unit needs to be positioned, and overall system cost is reduced along with resource needs (e.g., electrical power). The described systems and methods also enable the use of lower cost projectors that are separated from the single image capture unit (e.g., the projector can be located closer to the patient). The location of the projector does not need to be registered with the system.

In an aspect, a system includes a projector configured to project a pattern of dots within a tracking volume, a medical instrument having one or more markers, the medical instrument being positioned within the tracking volume, an image capture unit configured to capture imagery of the medical instrument and the one or more markers and configured to capture imagery of the pattern of dots within the tracking volume, and a computing device including a memory configured to store instructions and a processor to execute the instructions to perform operations. The operations include initiating capture, by the image capture unit, of at least two images of the medical instrument and the one or more markers, determining a three-dimensional position of the one or more markers from the captured images of the one or more markers, initiating projection, by the projector, of the pattern of dots within the tracking volume, initiating capture, by the image capture unit, of at least two images of a portion of the pattern of dots, and determining three-dimensional positions of dots in the portion of dots from the captured images of the portion of the pattern of dots.

Implementations may include one or more of the following features. The operations may include determining the three-dimensional position of the one or more markers and the three-dimensional positions of the dots in a same coordinate system. The operations may include tracking patient anatomy using the three-dimensional positions of the dots. Determining the three-dimensional positions of the dots may include using a portion of the dot pattern. Determining the three-dimensional positions of the dots may include determining a centroid. The operations may include matching the dots across the captured images of the portion of the pattern of dots. Projecting the pattern of dots may include projecting the pattern of dots in time intervals. Capturing the at least two images of the portion of the pattern of dots may be synchronized with the time intervals. The pattern of dots may be geometrically changed between subsequent projections. The image capture unit may include multiple cameras. The pattern of dots may include a pseudorandom pattern. Capturing the images of the one or more markers may occur during a first time period and capturing the images of the portion of the pattern of dots may occur during a second time period, wherein the first time period and the second time period are different. The projector may be mounted to a housing that contains the image capture unit. The projector may be positioned remote from a housing that contains the image capture unit. The projector may be portable.

In another aspect, a system includes a projector configured to project a pattern of dots within a tracking volume, wherein a medical instrument having one or more markers is positioned within the tracking volume, and an image capture unit including a memory configured to store instructions and a processor to execute the instructions to perform operations. The operations include initiating capture, by the image capture unit, of at least two images of the medical instrument and the one or more markers, determining a three-dimensional position of the one or more markers from the captured images of the one or more markers, initiating projection, by the projector, of the pattern of dots within the tracking volume, initiating capture, by the image capture unit, of at least two images of a portion of the pattern of dots, and determining three-dimensional positions of dots in the portion of dots from the captured images of the portion of the pattern of dots.

In another aspect, a method includes projecting, by a projector, a pattern of dots within a tracking volume, wherein the tracking volume further includes a medical instrument having one or more markers and the medical instrument is positioned within the tracking volume. The method includes capturing, by an image capture unit, at least two images of the medical instrument and the one or more markers, wherein the image capture unit is configured to capture imagery of the medical instrument and the one or more markers and configured to capture imagery of the pattern of dots within the tracking volume. The method includes determining, by a computer device, a three-dimensional position of the one or more markers from the captured images of the one or more markers and projecting, by the projector, of the pattern of dots within the tracking volume. The method includes capturing, by the image capture unit, of at least two images of a portion of the pattern of dots, and determining three-dimensional positions of dots in the portion of dots from the captured images of the portion of the pattern of dots.

Implementations may include one or more of the following features. The operations may include determining the three-dimensional position of the one or more markers and the three-dimensional positions of the dots in a same coordinate system. The operations may include tracking patient anatomy using the three-dimensional positions of the dots. Determining the three-dimensional positions of the dots may include using a portion of the dot pattern. Determining the three-dimensional positions of the dots may include determining a centroid.

The details of one or more embodiments of the subject matter described herein are set forth in the accompanying drawings and the description below. Other features, objects, and advantages of the subject matter will be apparent from the description and drawings, and from the claims.

### BRIEF DESCRIPTION OF THE DRAWINGS

FIG. 1 is a block diagram of an example tracking system using markers.

FIG. 2 is a block diagram of an example tracking system using marker and markerless techniques.

FIG. 3 is a computer system executing a tracker that processes received data and determines a three-dimensional (3D) position.

FIG. 4A is a diagram of an image capture unit that can be used in a tracking system.

FIG. 4B is a diagram of a projector that can be used in the tracking system of FIG. 2.

FIG. 5 is a diagram of dots that can be projected by the projector of FIG. 4B.

FIG. 6 is a series of images that represent dots projected on a patient.

FIG. 7 is a diagram of multiple dot patterns with different orientations being projected on a patient.

FIG. 8 is a diagram of a point cloud from one projection of dots and a point cloud from multiple projections of dots.

FIG. 9 is a diagram of centroids being matched across imagery of dot patterns.

FIG. 10 is a diagram of a point cloud being converted into a 3D anatomy of a patient.

FIG. 11 is a flowchart of operations of a tracker.

FIG. 12 is a diagram of an example computing system.

Like reference numbers and designations in the various drawings indicate like elements.

### DETAILED DESCRIPTION

Various types of tracking systems (e.g., optical, electromagnetic, etc.) can be employed for tracking objects (e.g., medical instruments in a surgical theater) in which markers are affixed to an exterior surface of the tracked object. For example, an object can include a marker that provides a signal that can indicate the position and orientation (e.g., pose) of the object in an environment (e.g., a tracking volume). The tracking system can be an optical tracking system, and a passive marker configured to reflect an optical signal can be affixed to an object. For example, the marker can include a retroreflective coating that reflects an optical signal along a parallel path back towards a source of the optical signal. Such reflective coatings can include reflective beads (e.g., glass microspheres, plastic microprisms, etc.), various materials (e.g., having crystalline structures, etc.), etc.

Markerless systems can also be utilized; for example, a projector projects dots onto a patient for producing individual data points for tracking. For example, the projector can project dots as, e.g., infrared light, near infrared light, visual dots, using different portions of the electromagnet spectrum, etc. While this disclosure describes dots being projected onto patients, other types of objects can have the dots projected upon them. The projector can be a low cost projector, but system processing can create high quality data from the low cost projector. In this way, a low cost projector can be utilized while not sacrificing data accuracy, e.g., in surgical environments.

The same image capture unit (e.g., that can be positioned, moved, etc. as a single unit) can be used to capture both the dots and the reflected optical signal from the marker (or markers). Various information can be attained from the captured images. In this particular environment, the tracking system is configured to estimate where the object (e.g., the medical instrument) is relative to the patient based on the reflected signal (from the markers) and the dots. For example, the patient data attained from the projected dots can provide a reference for the object data. By using these data sets, the patient data and the object data can be tracked in a common coordinate system.

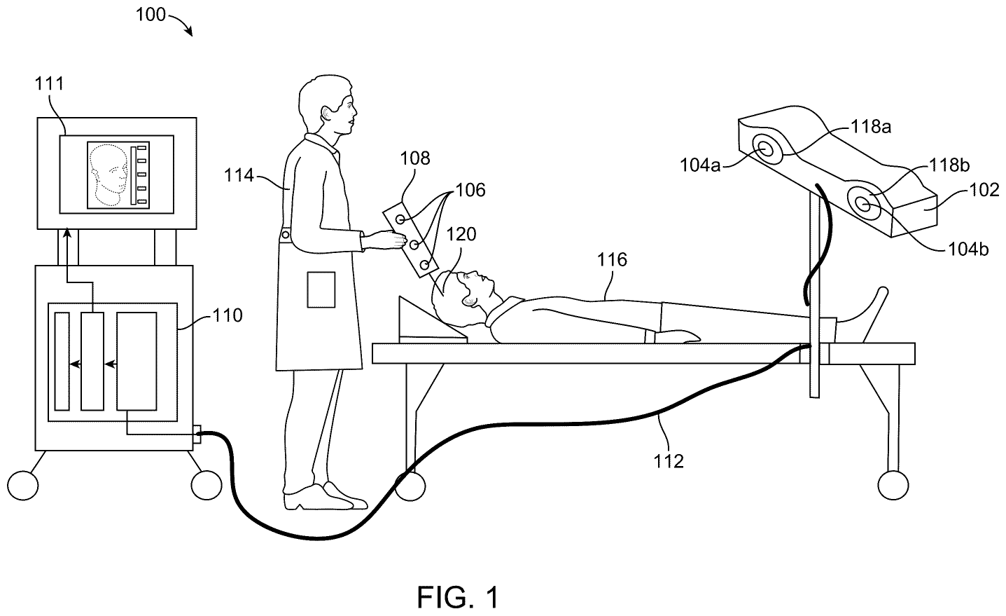

Referring to FIG. 1, an example tracking system 100 is illustrated that includes an illumination/image capture unit 102 in which a marker sensing device (e.g., a camera, an array of cameras 104a-b, etc.) and marker illuminating device(s) 118a-b (e.g., electromagnetic waves source) that are rigidly mounted. In this example, the illuminating devices 118a-b emit electromagnetic waves within one or more portions of the electromagnetic spectrum (e.g., radio frequency signals, visual signals, infrared signals, etc.). The electromagnetic waves are directed at a region that includes one or more markers 106 (e.g., retroreflective markers) that are affixed (e.g., rigidly affixed) to an object. In the context shown in FIG. 1, the object can be a tool 108 (e.g., a surgical tool, medical device for treating a patient, etc.) to which there is an interest in having the object tracked. In this example, the markers 106 are configured to have retro-reflectivity to reflect incoming electromagnetic waves in a parallel and in a direction opposite the direction of the incident waves. In this exemplary system, the cameras 104a-b capture one or more images of the illuminated markers 106. Due to the highly retro-reflective nature of the markers 106, each marker appears as a relatively bright spot in the captured images, and the system can determine the spatial coordinates (e.g., Cartesian, spherical, cylindrical, etc.) of the markers and an intensity value that represents, for example, the brightness of each corresponding reflected spot. One or more techniques can be employed to determine spatial coordinates, etc. For example, a computer system 110 is included in the system 100 that executes operations to determine spatial coordinates of the markers. The computer system 110 can include a display 111 configured to display images, coordinates, etc. The computer system 110 can determine the 3D position of the markers, 106, e.g., by analyzing the images to identify positions of the markers 106 in the images for which image coordinates (e.g., {U, V}, {row, column}, etc.) are calculated to sub-pixel resolution.

These image coordinates, such as {U, V} coordinates, from two or more cameras are used to compute the 3D position of the markers in a coordinate system (e.g., a Cartesian “XYZ” coordinate system). For example, the {U, V} coordinates can be processed to generate 3D positions from multiple stereoscopic images (e.g., through triangulation of the location of the cameras 104a-b and the location of the markers 106).

For example, the tracking techniques employed for tracking markers may be similar to those described in U.S. patent application Ser. No. 17/529,881, entitled “ERROR COMPENSATION FOR A THREE-DIMENSIONAL TRACKING SYSTEM”, filed on Nov. 18, 2021, which is hereby incorporated by reference in its entirety.

For efficient image processing, the system can be designed so that the markers provide very high contrast images, i.e., the markers are very bright relative to the rest of the image. This high contrast is usually achieved by using a retro-reflective material that strongly reflects electromagnetic waves emitted from the illumination devices.

To be provided data, the computer system 100 is connected to other system components; for example, the computer system 110 is connected to the array of cameras 104a-b via communication connections 112 (e.g., wired communication links, wireless communication connections, combinations of connections, etc.). Similarly, various types of connections can be employed to allow the computer system 110 to share information; for example, various connections can be used for sharing data with one or more networks. Along with different types of connections, various types of computer systems can be utilized; for example, stand-alone computers (as illustrated in the figure) can be used or the computer system can be combined with other system components (e.g., the computer can be combined with the image capture unit 102).

Various types of computer systems can also be used; for example, laptops, desktops, workstations, servers, blade servers, mainframes, etc. The computer system 110 can be realized by a distribution of computer systems; for example, one or more mobile computing devices (e.g., laptops, tablet computing devices, smartphones, etc.) can be used in combination with a stand along computing device (e.g., a server) to execute operations in a distributed manner and attain determinations. The components shown here, their connections and relationships, and their functions, are meant to be examples only, and are not meant to limit implementations of the techniques described and/or claimed in this document.

Given the known locations of the cameras 104a-b included in the array and the locations of the markers 106, the computer system calculates a 3D position of the object 108. Further, on the basis of the known relationship between the location of each of the markers 106 and the location of a tip 120 of the object 108 in the working volume (e.g., a tool coordinate system), the computer system calculates the coordinates of the tool tip 120 in space. In those instances in which the tool 108 is handled by a user (e.g., a surgeon 114) and the tool tip 120 is pressed against or is otherwise in contact with a surface (e.g., a body 116 of a patient), the coordinates of the tool tip 120 correspond to the coordinates of the point at which the tool tip 120 contacts the surface. In some implementations, the computer system can calculate an orientation of the object 108, e.g., given a known relationship between the location of each of the markers 106 on the object 108.

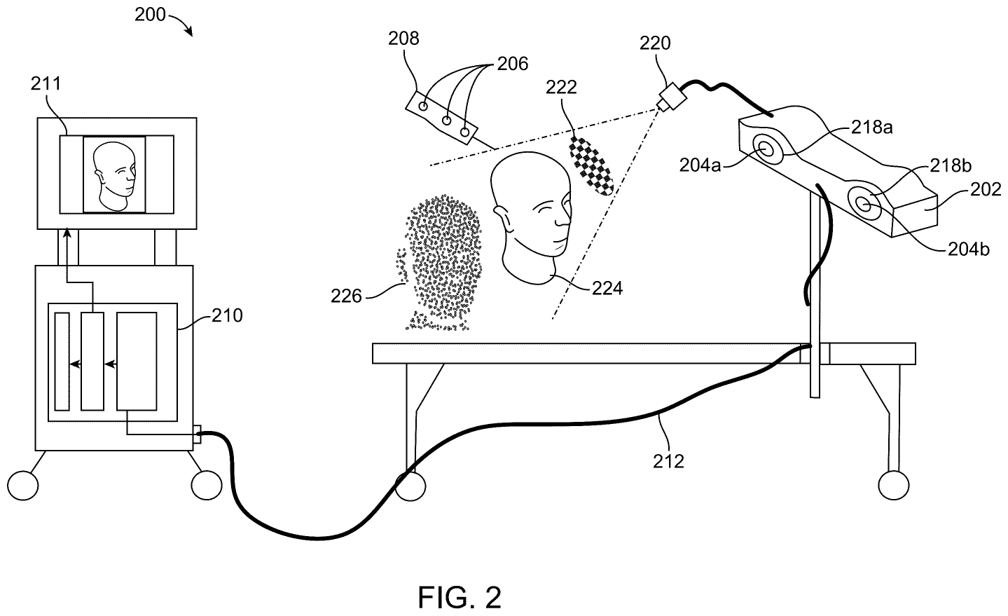

Referring to FIG. 2, an example tracking system 200 is presented that employs a markerless tracking technique along with a marker tracking technique (e.g., similar to the marker tracking system of FIG. 1). For example, employing a markerless tracking technique in combination with a marker tracking technique can allow for tracking of a tool (e.g., having markers) relative to a patient (e.g., not having markers). In the illustrated example, the system 200 includes an illuminator/image capture unit 202 that provides two capabilities: a capture unit (e.g., a camera, an array of cameras 204a-b, etc.) and an illumination device (e.g., illuminating device(s) 218a-b). With reference to FIG. 1, the illuminator/image capture unit 202 can be used for capturing imagery, e.g., one or more images, of markers for marker-based tracking.

The tracking system 200 also includes a projector 220 that can project dots (e.g., a pattern of dots 222) upon a portion of a patient 224 (e.g., a portion of a patient's head). The patient 224 does not have markers, but the tracking system 200 can track the patient 224 using the projected visuals. The projected pattern of dots 222 creates a representation that forms a point cloud 226 that represents various geometries, shapes, etc. of the one or more surfaces being projected upon (e.g., surfaces of the portion of the patient's head). Once the pattern of dots 222 is projected, the illuminator/image capture unit 202 can capture one or more images of the pattern of dots 222 (e.g., using the cameras 204a-b) and the captured imagery, e.g., a set of images or multiple sets of images, can be provided to a computer system 210.

Similar to the computer system 110 of FIG. 1, various types of computing devices and computing architectures can be employed along with different techniques for communicating with other system components. Various type of information can be determined from the captured images, for example, the computer system 210 can create a numerical representation of each dot represented in the point cloud 226, determine the 3D position (e.g., represented in one or more coordinate systems) of each dot represented in the point cloud, etc. Various information provided by the point cloud can be used by the computer system 210; for example, one or more parameters (e.g., intensity) of the dots (in the captured images) represented in the point cloud can be used to determine the 3D position of each dot. Similar to processing dot-level information, portions of the dots can be processed as described below. In other implementations, the projector 220 can project the dots 222 upon other objects (e.g., medical instruments). The computer system 210 also includes a display 211 configured to display images, coordinates, etc.

Along with being used to capture imagery, including a stream of images, to represent a point cloud, the illuminator/image capture unit 202 is utilized for a marker based tracking, e.g., to track a tool having one or more attached markers. From the captured imagery, a computer system 210 can determine information regarding the one or more markers; for example, the 3D position (e.g., represented in one or more coordinate systems) and an intensity value that represents, for example, the brightness of each corresponding marker. From this information, the computer system 210 can determine the position of the markers 206 with respect to a coordinate system. For example, the computer system 210 can determine the 3D position of the tool and the 3D position of each of the dots in the same coordinate system. This can be advantageous because the position and orientation of the tool and the position, anatomy, orientation, etc. of the patient can easily be determined relative to each other. For example, the 3D position of the tool and the 3D position of the patient can easily be determined in a common coordinate system (e.g., without co-registration of multiple components) because the images of the tool and the images of the dots are captured by the same image capture unit.

Being used for both marker and marker-less tracking, functionality of a single device, i.e., the illuminator/image capture unit 202, is used to execute both operations. For example, the illuminator/image capture unit 202 is used to capture images containing the dots 222 and images containing the illuminated markers 206. Various capture techniques can be employed for collecting the imagery; for example, the marker imagery, e.g., one or more images of markers, and dot pattern imagery, e.g., one or more images of dot patterns, can be collected during the same time period, during overlapping time periods, adjacent time periods, etc. In one implementation, the illumination capability of the illuminator/image capture unit 202 is used for collecting both sets of imagery. In some implementations, the illuminator/image capture unit 202 captures marker imagery and dot pattern imagery during separate and distinct time periods. For example, a dot pattern image can be collected during a time period that is between two time periods during which marker images are collected.

Other types of image capture sequences may also be employed by the illuminator/image capture unit 202 along with different capture patterns (e.g., capture a pair of marker images followed by a pair of dot pattern images, etc.), capture frequencies, etc. For one particular example, during one time period, the illuminator/image capture unit 202 captures images of the illuminated markers 206 while the pattern of dots 222 is not being projected by the projector 220. During a separate second time period, the capture unit 202 captures images of the pattern of dots 222, but not the illuminated markers 206. For example, the capture unit 202 can capture images of the dot patterns 222 while the markers 206 are not illuminated by the illuminating devices 218a-b. For example, different light signals with different frequencies, wavelengths, etc. can be used to illuminate the markers 206, such that the markers 206 are not illuminated when the dot pattern 222 is projected. By executing image captures (for the markers 206 and the dot pattern 222) during different time periods, the interference between the visibility of the markers 206 and the dot pattern 222 can be reduced.

The computer system 210 can determine the 3D position of the markers and the 3D position of the dots from the captured imagery. For example, the computer system can analyze the images of the markers to identify positions of the markers by converting image coordinates (e.g., {U, V}, {row, column}, etc.) into the 3D position of the markers in a coordinate system (e.g., a Cartesian “XYZ” coordinate system) as described above. The computer system can also analyze the images of the pattern of dots to identify 3D positions of individual dots within the pattern of dots, e.g., using triangulation, by converting image coordinates (e.g., {U, V}, {row, column}, etc.) into the 3D position of the dots in a coordinate system (e.g., a Cartesian “XYZ” coordinate system) as described above. Other techniques of analyzing images to identify 3D positions of the dots can also be utilized. Identifying 3D positions of individual dots is also further discussed below.

Given the known locations of the cameras 204a-b included in the array and the image coordinates of the markers 206, the computer system can calculate a 3D position of the tool 208, e.g., as discussed above with reference to FIG. 1. Further, on the basis of the known relationship between the location of each of the markers 206 and the location of a tip of the tool 208 in the working volume (e.g., a tool coordinate system), the computer system calculates the 3D position of the tool tip in space. In those instances in which the tool 208 is handled by a user and the tool tip is pressed against or is otherwise in contact with the patient 224, the 3D position of the tool tip corresponds to the 3D position of a part of the patient 224. Data representing the position of the tool 208 and data representing the position of the patient 224 (attained from the point cloud 226) can be registered in a common coordinate system, so that the tool and the patient can be tracked relative to each other.

The same image capture unit of the illuminator/image capture unit 202 can capture images of the pattern of dots 222 and of the markers 206. Using the same illuminator/image capture unit 202 to capture both sets of images is advantageous because it reduces the number of components in the system for the end user. For example, the computer system can calculate a 3D position of the patient using a markerless technique (e.g., as described above) given the known location of the illuminator/image capture unit 202, and the computer system can also calculate the 3D positions of the markers 206 using a marker technique (e.g., as described above) given the known location of the image capture unit 202. Since there is only a singular illuminator/image capture unit 202, the computer system can calculate the 3D positions of the markers 206 and the 3D positions of the dots (representing the patient 224) from the same reference (e.g., the known location of the illuminator/image capture unit 202). In contrast, using multiple illuminator/image capture units would require co-registration of the positions and orientations of the multiple illuminator/image capture units. Also, using a single illuminator/image capture unit reduces the cost of the system.

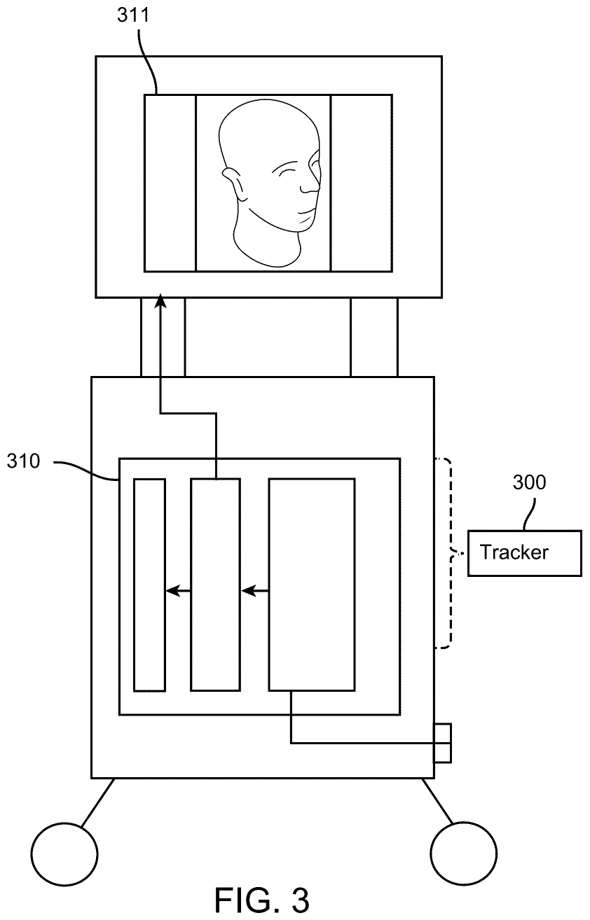

The computer systems described can execute operations (e.g., an application program) referred to as a tracker to determine the 3D position and orientation of the tool and the 3D position of each dot in the dot pattern. For example, the tracker can utilize the captured data to determine a 3D position of a surgical tool (or other object) and a 3D position of a patient, patient anatomy, etc., e.g., using marker or markerless techniques described above. Referring to FIG. 3, in this illustrated example a computer system 310 (e.g., similar to computer system 110 or computer system 210) executes a tracker 300 that can be implemented in hardware, software, a combination of hardware and software, etc. Software implementations, (e.g., a program, an application, etc.) typically includes executable instructions for a programmable processor, and can be implemented in high-level procedural techniques (e.g., using object-oriented programming language), lower-level techniques (e.g., assembly or machine language), etc. Similar to the computer system 110 of FIG. 1 or the computer system 210 of FIG. 2, the computer system 310 includes a display 310 configured to display images, coordinates, and other types of data related to the location of the markers.

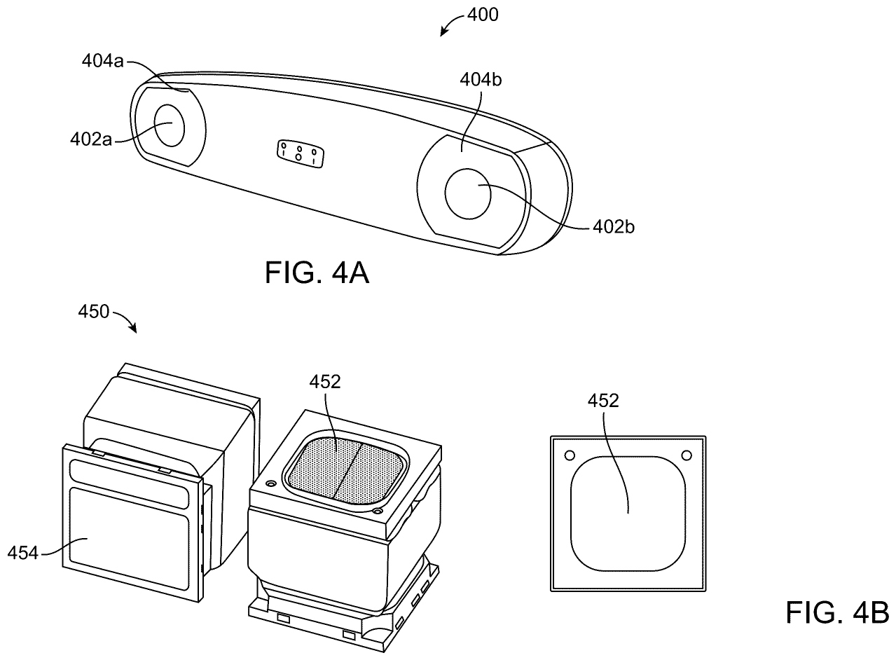

By employing a single illuminator/image capture unit, the location of one illuminator/image capture unit (rather than multiple units) needs to be registered with the system. Further, only a single illuminator/image capture unit needs to be positioned, and overall system cost is reduced along with resource needs (e.g., electrical power). A variety of illuminator/image capture units can be used in the systems described above. Referring to FIG. 4A, an exemplary illuminator/image capture unit 400 includes an array of cameras 402a-b and illuminating devices 404a-b. The illuminating devices 404a-b can emit electromagnetic waves, such as visible light, infrared light, etc. The array of cameras 402a-b can act as a marker sensing device, as described above. The image capture unit 400 can be used similarly to the image capture unit 102 of FIG. 1, similarly to the image capture unit 202 of FIG. 2, etc. In some implementations, the illuminating devices can be separate from the image capture unit (e.g., separate from a housing of the image capture unit).

A variety of projectors can be used in the systems described above. For example, the projector 220 is external to the illuminator/image capture unit 202 in FIG. 2. In some implementations, the projectors can be located near the illuminator/image capture unit (e.g., mounted to a housing of the illuminator/image capture unit) or included in the image capture unit (e.g., contained within the housing of the illuminator/image capture unit). Referring to FIG. 4B, an exemplary projector 450 includes a projection face 452. The projector 450 is depicted as having a cuboid geometry, but in other implementations can be other geometries (e.g., a prism geometry, a spherical geometry, etc.). The projector 450 can have dimensions of approximately 3.50 inches by 3.50 inches by 3.40 inches. The projection face 452 can have dimensions of approximately 2.5 inches by 2.5 inches. In other implementations, the projection face 452 can have the same dimensions as one side of the projector 450. In other implementations the projector 450 can be larger (e.g., 10 inches by 10 inches by 10 inches) or smaller (e.g., 2 inches by 2 inches by 2 inches). The projector 450 can also include a mounting face 454. For example, the mounting face can provide a flat surface for the projector 450 to be mounted to another object (e.g., a camera, a housing of the illuminator/image capture unit, etc.).

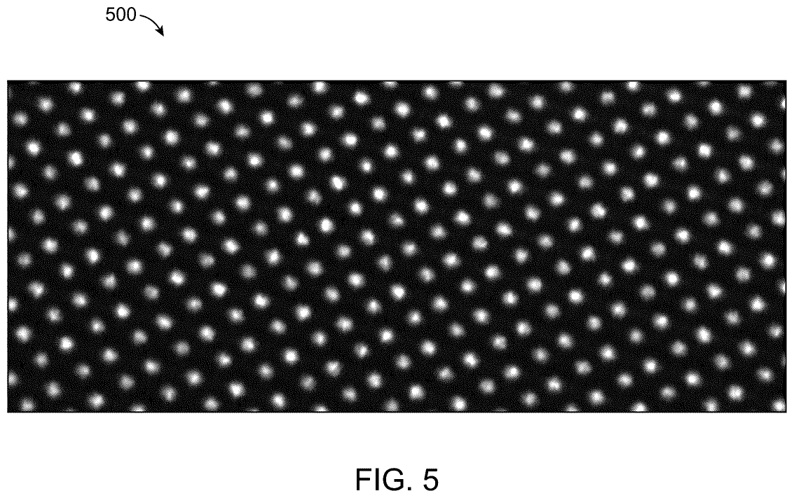

Various patterns of dots can be projected by the projectors to track an object. Referring to FIG. 5, an exemplary pattern of dots 500 includes a uniform pattern of dots. For example, each dot is equidistant from the surrounding dots. The pattern of dots 500 can be projected from a projector, as described above with reference to FIG. 2. In some embodiments, the pattern of dots is not uniform. For example, the pattern of dots can have a random pattern (e.g., by randomizing the pattern of dots) or a pseudorandom pattern (e.g., as described further below).

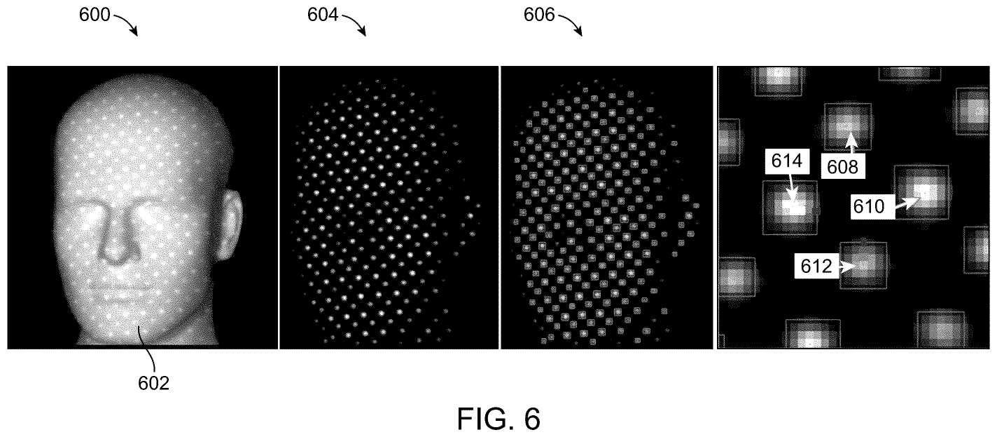

The pattern dots that are projected onto a patient can be analyzed to determine a position and orientation of the patient (e.g., the patient's body part, face, head, etc.). FIG. 6 illustrates a pattern of dots 600 being projected onto a patient 602. The dots 600 include a uniform pattern of dots. The dots 600 can be projected from a projector, as described above with reference to FIG. 2. The pattern of dots 600 creates a point cloud of the patient 602. For example, point cloud 604 is composed of dots from the pattern of dots 600 and can represent the shape, position, orientation, etc. of the patient 602. An image of the point cloud 604 can be captured by an image capture unit (e.g., the illuminator/image capture unit 102 of FIG. 1, the illuminator/image capture unit 202 of FIG. 2, etc.). In some implementations, the projector projects the dots 600 in time intervals which are synchronized with the image capturing by the image capture unit. For example, synchronizing the projector with the image capturing by the image capture unit can allow for increased intensity and brightness of the projection because more power can be used for a shorter duration. This makes the projection brighter for image capturing and can increase the accuracy of the projection. In other implementations, the projector is not synchronized with the image capturing by the image capture unit. The intervals of projections can be uniform or dynamic. In some implementations, the projector does not project the pattern of dots 600 in time intervals.

The image capture unit can transmit the captured imagery, including one or more images, sets of images, or some combination thereof, to a computer system (e.g., the computer system 110 of FIG. 1, the computer system 210 of FIG. 2, etc.). In some cases, the one or more images can include a stream of images at different time instances, multiple images at the same time instance (e.g., from multiple cameras in the image capture unit, multiple image capture units), or some combination thereof. The computer system can calculate dot segment information about each dot in the point cloud 604. For example, the computer system can calculate the centroid (e.g., center of mass) of each dot in the point cloud 604. For example, the point cloud 606 can represent an intermediate step in which the computer system calculates the centroid of each dot in the imagery captured by the image capture unit. As a non-limiting example, the centroids 608, 610, 612, 614 are illustrated. In another example, the dot segment information can include, e.g., a center of area of a dot. In some implementations, the computer system does not calculate dot segment information of every dot and only calculates dot segment information of a portion (e.g., half, third, etc.) of the dots. In some implementations, the computer system calculates dot segment information of each dot that is visible in the captured imagery. The dot segment information can be calculated, e.g., by segmenting an image of the dots into pixels and/or subpixels. A portion of the dot pattern, e.g., a number of the dots, can be utilized to determine the dot segment information. For example, multiple dots can be used to triangulate the dot segment information. The dot segment information (e.g., the centroid 608) of a dot can be calculated, e.g., using a center of mass formula. In another example, a center of area can be calculated, e.g., using a center of area formula. The dot segment information (e.g., the centroid 608, the center of area, etc.) can be converted into a set of 3D coordinates (e.g., representing a 3D position) that is stored by the computer system. For example, image analysis can be used to identify 3D positions of the dots in the images for which image coordinates (e.g., {U, V}, {row, column}, etc.) are calculated to compute the 3D position of the dots in a coordinate system (e.g., a Cartesian “XYZ” coordinate system), e.g., as described above. For example, the {U, V} coordinates can be processed to generate 3D positions from multiple stereoscopic images (e.g., through triangulation of the location of the cameras 104a-b and the image coordinates of the dots).

Using dot segment information of each projected dot reduces the total number of data points in the point cloud (e.g., when compared against a pixel matched disparity depth map), but can increase accuracy due to increased sub-pixel resolution. This can create high resolution data from captured images of a low-resolution projection, and can allow fixed pattern projectors with low resolution to achieve a high scanning accuracy (e.g., 0.01-0.1 pixels) even in a large volume. For example, this can allow a low cost projector to be used to track a patient (or other object) with high accuracy.

Other methods can be used in addition or alternatively to determining centroids to determine the 3D coordinates of individual dots. For example, pixel matching can be used to create a disparity map and determine 3D coordinates of dots. Pixel matching disparity maps can be created by matching pixels in a first image with corresponding pixels in a second image (e.g., using a stereo camera system). After matching the pixels, distance values can be combined with known camera geometries to determine a position of each pixel (e.g., via triangulation).

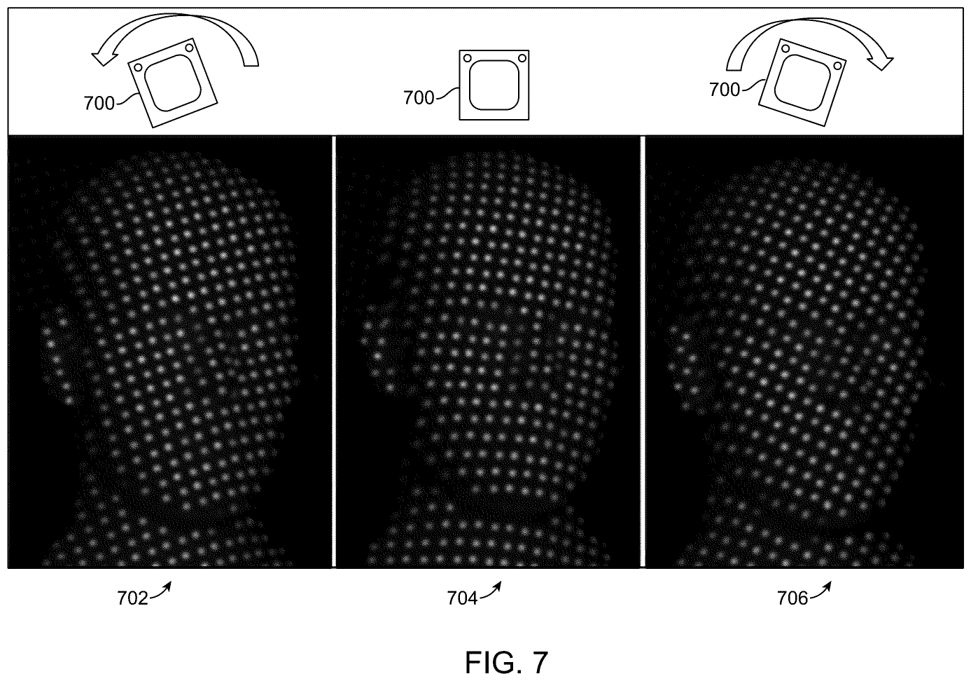

Increasing the number of dots can increase the accuracy of the tracking system (e.g., by increasing the amount of data captured by the tracking system). For example, FIG. 7 illustrates multiple projections of dots being projected on a patient. Each projection has a different orientation. In this illustrated example, a projector 700 can project a pattern of dots at a first orientation. For example, the projector 700 can project dots as described above. In the first orientation, the projected pattern creates a first point cloud 702 of the patient. The first point cloud 702 can represent the shape, position, orientation, etc. of the patient. The projector 700 can also project dots at a second orientation. In the second orientation, the pattern of dots creates a second point cloud 704 of the patient. The second point cloud 704 represents the shape, position, orientation, etc. of the patient differently from the first point cloud 702 because of the different orientation of projected pattern. The projector 700 can also project dots at a third orientation. In the third orientation, the pattern of dots creates a third point cloud 706 of the patient. The third point cloud 706 represents the shape, position, orientation, etc. of the patient differently from the first point cloud and the second point cloud. Any number of different orientations can be utilized to create additional representations of the patient. Each different orientation can provide additional data about the features of the patient.

In some implementations, multiple (e.g., two, three, four, etc.) projectors can have different orientations to provide different point clouds. In other implementations, a single projector can project dot patterns having different orientations. For example, a single projector can project a pattern of dots in time intervals. Each projection emitted by the projector can have a different orientation. In some implementations, the different orientations can be created from data provided to the projector, such that the projector creates different projections (e.g., different patterns, different orientations, etc.). In some implementations, the projector itself can change orientations in between projections to provide different point clouds. In some implementations, the time intervals are synchronized with image capturing by an image capture unit. For example, synchronizing the projector with the image capturing increases the intensity and brightness of the projection because more power can be used for a shorter duration. This makes the projection brighter for image capturing and can increase the accuracy of the projection. In some implementations, the projection can be geometrically changed (e.g., rotated, moved, etc.) in between projections so that each projection provides a different point cloud. Each projection can provide a different point cloud of the same object, e.g., because the projections have different orientations.

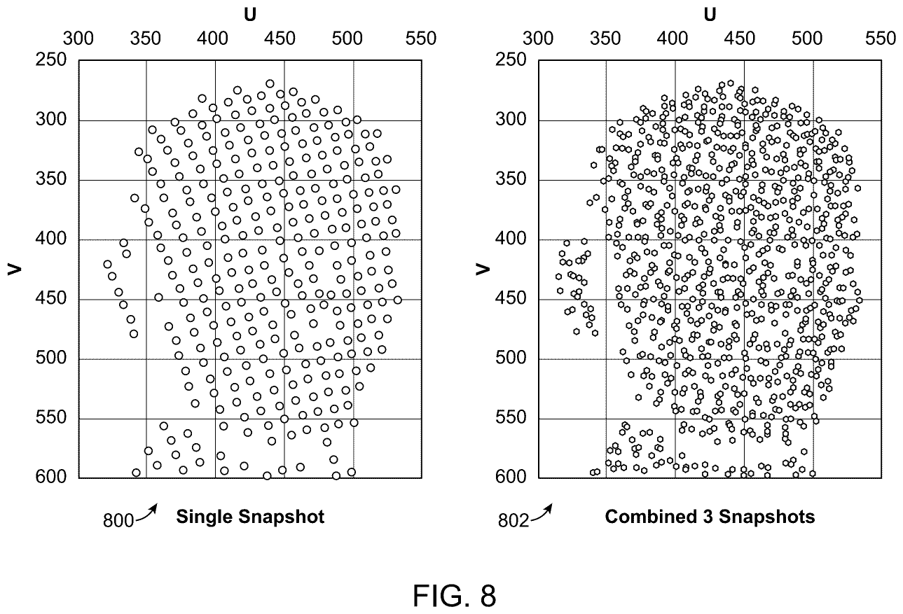

Multiple projections of dots (e.g., projections having different orientations) can be put together to create a more accurate representation of the patient. For example, FIG. 8 illustrates a point cloud from one projected pattern of dots and a point cloud from multiple projected patterns of dots. The multiple projected patterns of dots increase the number of dots captured by the system and increase the accuracy of the system. The multiple projected patterns can be combined to form a pseudorandom pattern. A first point cloud 800 is representative of a single pattern of dots projected on a patient. A second point cloud 802 is representative of three patterns of dots projected on the same patient, e.g., to form a pseudorandom pattern. For example, each of the three patterns can have a different orientation, as described above with reference to FIG. 7. As illustrated, second point cloud 802, i.e., from the combination of the three patterns of dots, is a denser point cloud than the first point cloud 800, i.e., from the single pattern of dots. The second point cloud 802 also has a pseudorandom pattern. The second point cloud 802 provides more data representing the shape, position, orientation, etc. of the patient. Capturing more data will create a better representation of the patient and provide tracking with higher accuracy.

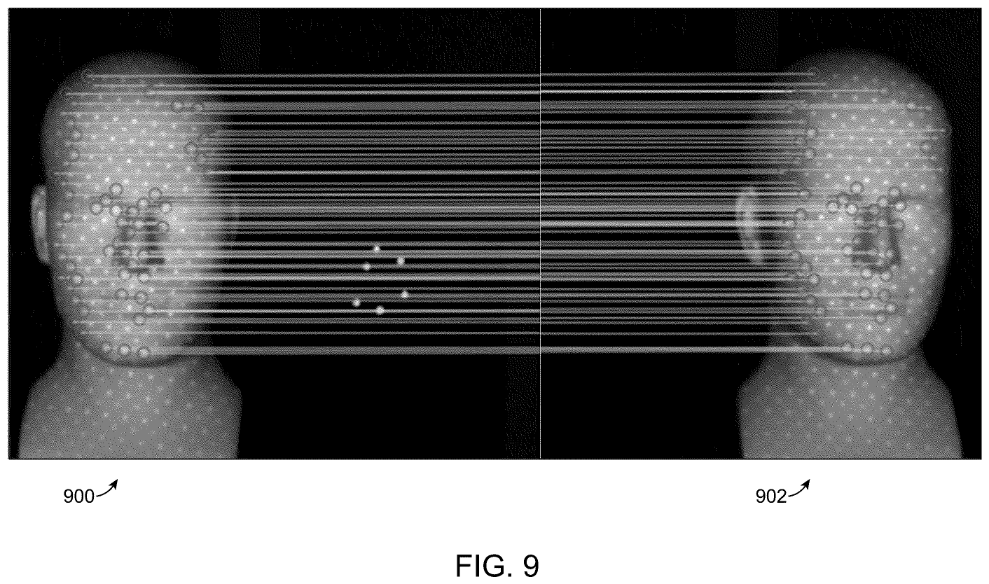

When multiple images are captured of dots being projected on an object, individual dots (or dot segment information) of the dots can be matched across the multiple images to increase the accuracy of the representation of the patient. For example, in some implementations multiple images are captured of the same dot pattern, e.g., using an image capturing unit with multiple cameras, multiple image capturing units, etc. When multiple images are captured of the same pattern of dots, it can be advantageous to match corresponding dots and dot segment information across the multiple images. FIG. 9 illustrates dots being matched across different images. A first captured image 900 contains a pattern of dots being projected on a patient. A second captured image 902 contains the same pattern of dots being projected on the same patient. The second captured image 902 can be captured from a slightly different angle, e.g., due to positioning of multiple cameras, multiple image capture units, etc. At least a portion of the pattern of dots appear in both images 900, 902. A dot which appears in both images 900, 902 can be matched across the images to increase the overall accuracy of the tracking. For example, corresponding dot locations can be triangulated using known camera geometries. Additionally or alternatively, the intensity/brightness of each dot can be used to match dots (or dot segments) across the images. In some implementations, infrared (IR) lighting can highlight the patient, and the dots can be compared to their location in an IR image to match the centroids across the images. In some implementations, the dots can be matched across the images using calculated geometries of the pattern of dots (e.g., angles and distances between the dots).

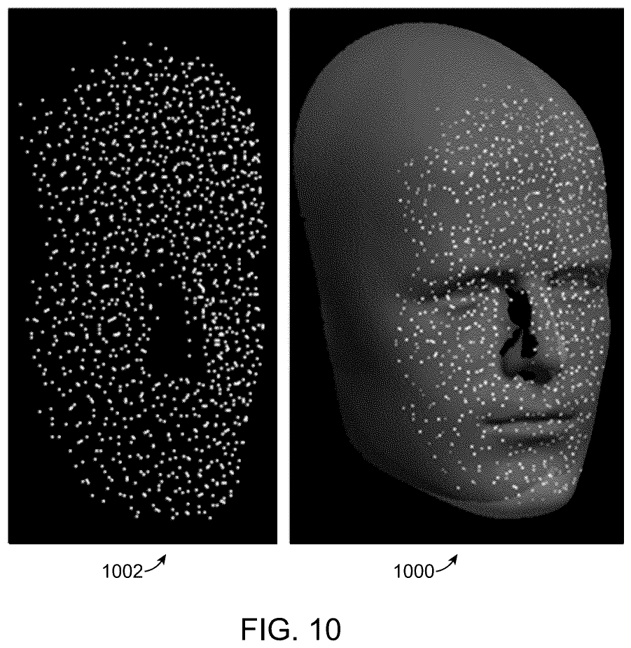

Captured images can be processed by a computer system to calculate 3D positions of each dot. FIG. 10 illustrates 3D positions of dots calculated from a point cloud 1002. For example, the point cloud 1002 can be representative of the face of a patient 1000. In implementations where the dot represents a patient, the 3D positions can be presented to a medical professional (e.g., a surgeon) to visualize the position of a tracked tool (e.g., a surgical tool, medical device for treating a patient, etc.) relative to the 3D position of the dots (e.g., relative to the patient). For example, in the system 200 of FIG. 2 above, the computer system 210 is configured to determine where in the environment the markers and the tool 208 are in the coordinate system and with respect to the patient 224. In some implementations, the 3D positions of the markers and the 3D positions of the dots are determined in a common coordinate system.

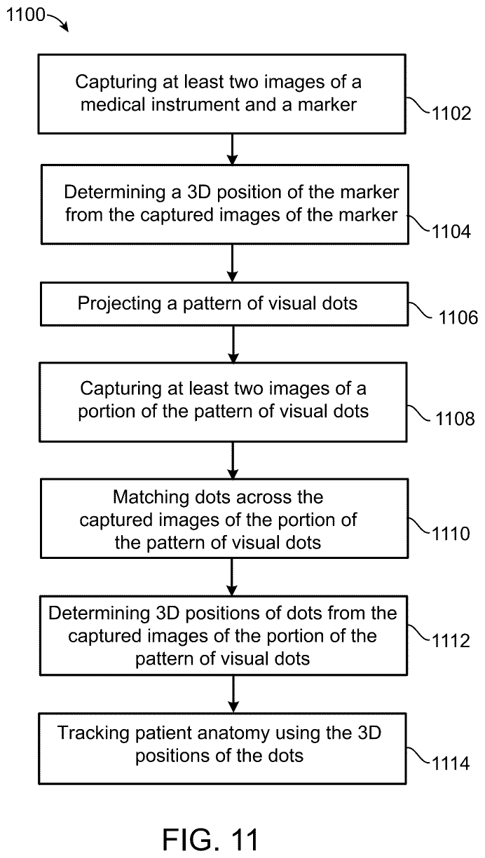

FIG. 11 is a flowchart for a method 1100 representing operations of a tracker. For example, the tracker can be similar to the tracker 300 of FIG. 3. The operations may include capturing at least two images of a medical instrument and a marker (1102). For example, the images can be captured using an image capture unit similar to the image capture unit 202 of FIG. 2.

The operations may further include determining a 3D position of the marker from the captured images of the marker (1104). For example, the 3D position of the marker can be determined, e.g., by analyzing the images to identify positions of the marker in the images for which image coordinates (e.g., {U, V}, {row, column}, etc.) are calculated to sub-pixel resolution. These image coordinates can be used to compute the 3D position of the marker in a coordinate system (e.g., a Cartesian “XYZ” coordinate system).

The operations may further include projecting a pattern of dots (1106). For example, a projector can project a pattern of dots upon a patient. The projector can be similar to the projector 220 of FIG. 2. In other implementations, the dots can be projected on other objects (e.g., a cadaver, a surgical table, etc.) within the tracking volume. In some implementations, the projector projects the dots in time intervals which are synchronized with image capturing by the image capture unit. For example, synchronizing the projector with the image capturing increases the intensity and brightness of the projection because more power can be used for a shorter duration. This makes the projection brighter for image capturing and can increase the accuracy of the projection. In other implementations, the projector is not synchronized with the image capturing. The time intervals of projections can be uniform or dynamic. In some implementations, the projector does not project the dots in intervals. In some implementations, the projections can be geometrically changed (e.g., rotated) to provide different orientations, e.g., similar to FIG. 7. The multiple projected patterns can be combined to form a pseudorandom pattern, e.g., similar to FIG. 8. The projected patterns can have other patterns (e.g., uniform, random, etc.).

The operations may further include capturing at least two images of a portion of the pattern of dots using the same image capturing unit as the image capturing unit that captures at least two images of the medical instrument and the marker (1106). For example, using a single image capturing unit to capture the images of the marker and to capture the images of the portion of the pattern of dots reduces the cost of the system and also creates a simpler system for the end user. For example, using a single image capturing unit eliminates the need to register the locations of multiple image capture units relative to each other.

The operations may further include matching dots across the captured images of the portion of the pattern of dots (1110). For example, dots which appear in multiple images can be matched across the images to increase the overall accuracy of the tracking. For example, corresponding centroid locations can be triangulated using known camera geometries.

Additionally or alternatively, the intensity/brightness of each dot can be used to match centroids across the images. In some implementations, infrared (IR) lighting can highlight the object, and the centroids can be compared to their location in an IR image to match the centroids across the images. In some implementations, the centroids can be matched across the images using calculated geometries of the pattern of dots (e.g., angles and distances between the dots).

The operations may further include determining 3D positions of dots from the captured images of the portion of the pattern of dots (1112). For example, the 3D positions of the dots can be determined, e.g., by analyzing the images to identify positions of the dots in the images for which image coordinates (e.g., {U, V}, {row, column}, etc.) are calculated to sub-pixel resolution. These image coordinates can be used to compute the 3D position of the dots in a coordinate system (e.g., a Cartesian “XYZ” coordinate system). For example, the coordinate system can be the same coordinate system in which the 3D position of the marker is computed. In some implementations, determining 3D positions of the dots includes calculating dot segment information the dots. For example, the dot segment information can be, e.g., the center of mass, the center of area, etc. For example, a dot can be segmented into pixels and/or subpixels. Then, the centroid can be calculated, e.g., using a center of mass formula. The dot segment information (e.g., the centroid) can be converted into a set of coordinates (e.g., 3D coordinates) as described above. Using dot segment information reduces the total number of data points (e.g., when compared against a pixel matched disparity depth map) but can increase accuracy due to increased sub-pixel resolution. This can create high resolution data from captured images of a low-resolution projection.

The operations may further include tracking the anatomy of the patient using the 3D positions of the dots (1114). For example, data representing the 3D position of the marker and data representing the 3D positions of the dots can be computed in a common coordinate system, so that the marker (and the medical instrument) is tracked relative to the dots. The 3D positions of the dots, the 3D position of the marker, etc. can be presented, e.g., on a display, to a medical professional (e.g., a surgeon) to visualize how the medical instrument moves relative to the pattern of dots. In some implementations, the pattern of dots can be projected on the medical instrument, such that the 3D position of the medical instrument is tracked using markerless techniques, as described above.

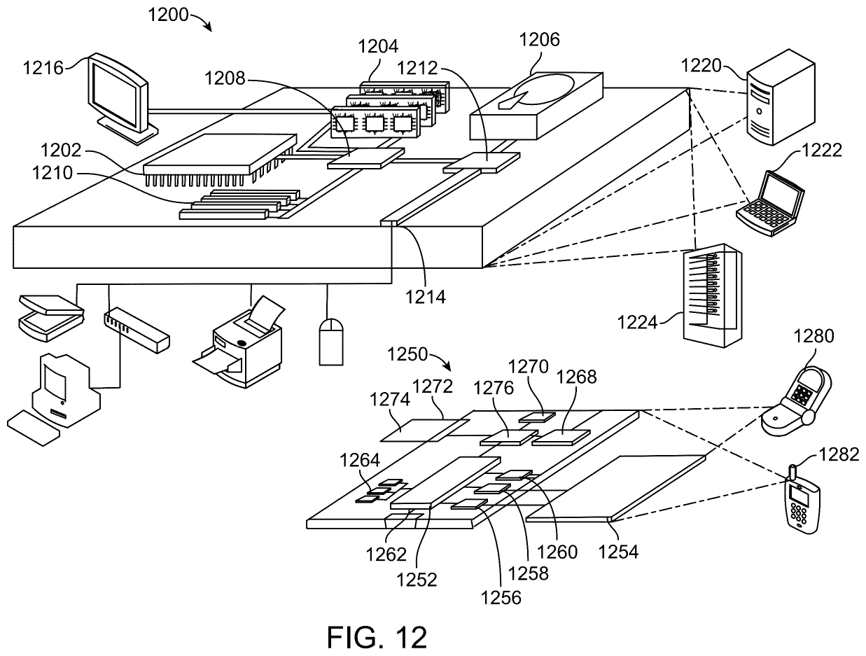

FIG. 12 shows an example computing device 1200 and an example mobile computing device 1250, which can be used to implement the techniques described herein. For example, the computing device 1200 may be implemented as the computing device 110 of FIG. 1 and/or the computing device 210 of FIG. 2. The computing device 1200 can be used to implement the method 1100 of FIG. 11. Computing device 1200 is intended to represent various forms of digital computers, including, e.g., laptops, desktops, workstations, personal digital assistants, servers, blade servers, mainframes, and other appropriate computers. Computing device 1250 is intended to represent various forms of mobile devices, including, e.g., personal digital assistants, cellular telephones, smartphones, and other similar computing devices. The components shown here, their connections and relationships, and their functions, are meant to be examples only, and are not meant to limit implementations of the techniques described and/or claimed in this document.

Computing device 1200 includes processor 1202, memory 1204, storage device 1206, high-speed interface 1208 connecting to memory 1204 and high-speed expansion ports 1210, and low-speed interface 1212 connecting to low-speed bus 1214 and storage device 1206. Each of components 1202, 1204, 1206, 1208, 1210, and 1212, are interconnected using various busses, and can be mounted on a common motherboard or in other manners as appropriate. Processor 1202 can process instructions for execution within computing device 1200, including instructions stored in memory 1204 or on storage device 1206, to display graphical data for a GUI on an external input/output device, including, e.g., display 1216 coupled to high-speed interface 1208. In some implementations, multiple processors and/or multiple buses can be used, as appropriate, along with multiple memories and types of memory. In addition, multiple computing devices 1200 can be connected, with each device providing portions of the necessary operations (e.g., as a server bank, a group of blade servers, a multi-processor system, etc.).

Memory 1204 stores data within computing device 1200. In some implementations, memory 1204 is a volatile memory unit or units. In some implementation, memory 1204 is a non-volatile memory unit or units. Memory 1204 also can be another form of computer-readable medium, including, e.g., a magnetic or optical disk.

Storage device 1206 is capable of providing mass storage for computing device 1200. In some implementations, storage device 1206 can be or contain a computer-readable medium, including, e.g., a floppy disk device, a hard disk device, an optical disk device, a tape device, a flash memory or other similar solid state memory device, or an array of devices, including devices in a storage area network or other configurations. A computer program product can be tangibly embodied in a data carrier. The computer program product also can contain instructions that, when executed, perform one or more methods, including, e.g., those described above. The data carrier is a computer-or machine-readable medium, including, e.g., memory 1204, storage device 1206, memory on processor 1202, and the like.

High-speed controller 1208 manages bandwidth-intensive operations for computing device 1200, while low-speed controller 1212 manages lower bandwidth-intensive operations. Such allocation of functions is an example only. In some implementations, high-speed controller 1208 is coupled to memory 1204, display 1216 (e.g., through a graphics processor or accelerator), and to high-speed expansion ports 1210, which can accept various expansion cards (not shown). In some implementations, the low-speed controller 1212 is coupled to storage device 1206 and low-speed expansion port 1214. The low-speed expansion port, which can include various communication ports (e.g., USB, Bluetooth®, Ethernet, wireless Ethernet), can be coupled to one or more input/output devices, including, e.g., a keyboard, a pointing device, a scanner, or a networking device including, e.g., a switch or router (e.g., through a network adapter).

Computing device 1200 can be implemented in a number of different forms, as shown in FIG. 12. For example, the computing device 1200 can be implemented as standard server 1220, or multiple times in a group of such servers. The computing device 1200 can also can be implemented as part of rack server system 1224. In addition or as an alternative, the computing device 1200 can be implemented in a personal computer (e.g., laptop computer 1222). In some examples, components from computing device 1200 can be combined with other components in a mobile device (e.g., the mobile computing device 1250). Each of such devices can contain one or more of computing device 1200, 1250, and an entire system can be made up of multiple computing devices 1200, 1250 communicating with each other.

Computing device 1250 includes processor 1252, memory 1264, and an input/output device including, e.g., display 1254, communication interface 1266, and transceiver 1268, among other components. Device 1250 also can be provided with a storage device, including, e.g., a microdrive or other device, to provide additional storage. Components 1250, 1252, 1264, 1254, 1266, and 1268, may each be interconnected using various buses, and several of the components can be mounted on a common motherboard or in other manners as appropriate.

Processor 1252 can execute instructions within computing device 1250, including instructions stored in memory 1264. The processor 1252 can be implemented as a chipset of chips that include separate and multiple analog and digital processors. The processor 1252 can provide, for example, for the coordination of the other components of device 1250, including, e.g., control of user interfaces, applications run by device 1250, and wireless communication by device 1250.

Processor 1252 can communicate with a user through control interface 1258 and display interface 1256 coupled to display 1254. Display 1254 can be, for example, a TFT LCD (Thin-Film-Transistor Liquid Crystal Display) or an OLED (Organic Light Emitting Diode) display, or other appropriate display technology. Display interface 1256 can comprise appropriate circuitry for driving display 1254 to present graphical and other data to a user. Control interface 1258 can receive commands from a user and convert them for submission to processor 1252. In addition, external interface 1262 can communicate with processor 1242, so as to enable near area communication of device 1250 with other devices. External interface 1262 can provide, for example, for wired communication in some implementations, or for wireless communication in some implementations. Multiple interfaces also can be used.

Memory 1264 stores data within computing device 1250. Memory 1264 can be implemented as one or more of a computer-readable medium or media, a volatile memory unit or units, or a non-volatile memory unit or units. Expansion memory 1274 also can be provided and connected to device 1250 through expansion interface 1272, which can include, for example, a SIMM (Single In Line Memory Module) card interface. Such expansion memory 1274 can provide extra storage space for device 1250, and/or may store applications or other data for device 1250. Specifically, expansion memory 1274 can also include instructions to carry out or supplement the processes described above and can include secure data. Thus, for example, expansion memory 1274 can be provided as a security module for device 1250 and can be programmed with instructions that permit secure use of device 1250. In addition, secure applications can be provided through the SIMM cards, along with additional data, including, e.g., placing identifying data on the SIMM card in a non-hackable manner.

The memory 1264 can include, for example, flash memory and/or NVRAM memory, as discussed below. In some implementations, a computer program product is tangibly embodied in a data carrier. The computer program product contains instructions that, when executed, perform one or more methods. The data carrier is a computer-or machine-readable medium, including, e.g., memory 1264, expansion memory 1274, and/or memory on processor 1252, which can be received, for example, over transceiver 1268 or external interface 1262.

Device 1250 can communicate wirelessly through communication interface 1266, which can include digital signal processing circuitry where necessary. Communication interface 1266 can provide for communications under various modes or protocols, including, e.g., GSM voice calls, SMS, EMS, or MMS messaging, CDMA, TDMA, PDC, WCDMA, CDMA2000, or GPRS, among others. Such communication can occur, for example, through radio-frequency transceiver 1268. In addition, short-range communication can occur, including, e.g., using a Bluetooth®, WiFi, or other such transceiver (not shown). In addition, GPS (Global Positioning System) receiver module 1270 can provide additional navigation-and location-related wireless data to device 1250, which can be used as appropriate by applications running on device 1250.

Device 1250 also can communicate audibly using audio codec 1260, which can receive spoken data from a user and convert it to usable digital data. Audio codec 1260 can likewise generate audible sound for a user, including, e.g., through a speaker, e.g., in a handset of device 1250. Such sound can include sound from voice telephone calls, recorded sound (e.g., voice messages, music files, and the like) and also sound generated by applications operating on device 1250.

Computing device 1250 can be implemented in a number of different forms, as shown in FIG. 12. For example, the computing device 1250 can be implemented as cellular telephone 1280. The computing device 1250 also can be implemented as part of smartphone 1282, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include one or more computer programs that are executable and/or interpretable on a programmable system. This includes at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to a computer program product, apparatus and/or device (e.g., magnetic discs, optical disks, memory, Programmable Logic Devices (PLDs)) used to provide machine instructions and/or data to a programmable processor, including a machine-readable medium that receives machine instructions.

To provide for interaction with a user, the systems and techniques described herein can be implemented on a computer having a display device (e.g., a CRT (cathode ray tube) or LCD (liquid crystal display) monitor) for presenting data to the user, and a keyboard and a pointing device (e.g., a mouse or a trackball) by which the user can provide input to the computer. Other kinds of devices can be used to provide for interaction with a user as well. For example, feedback provided to the user can be a form of sensory feedback (e.g., visual feedback, auditory feedback, or tactile feedback). Input from the user can be received in a form, including acoustic, speech, or tactile input.

The systems and techniques described here can be implemented in a computing system that includes a backend component (e.g., as a data server), or that includes a middleware component (e.g., an application server), or that includes a frontend component (e.g., a client computer having a user interface or a Web browser through which a user can interact with an implementation of the systems and techniques described here), or a combination of such backend, middleware, or frontend components. The components of the system can be interconnected by a form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In some implementations, the components described herein can be separated, combined or incorporated into a single or combined component. The components depicted in the figures are not intended to limit the systems described herein to the software architectures shown in the figures.

A number of embodiments have been described. Nevertheless, it will be understood that various modifications may be made without departing from the spirit and scope of the disclosure. Accordingly, other embodiments are within the scope of the following claims.

What is claimed is:

1. A system comprising:
a projector configured to project a pattern of dots within a tracking volume;
a medical instrument having one or more markers, the medical instrument being positioned within the tracking volume;
an image capture unit configured to capture imagery of the medical instrument and the one or more markers and configured to capture imagery of the pattern of dots within the tracking volume; and
a computing device comprising a memory configured to store instructions and a processor to execute the instructions to perform operations comprising:
initiating capture, by the image capture unit, of at least two images of the medical instrument and the one or more markers;
determining a three-dimensional position of the one or more markers from the captured images of the one or more markers;
initiating projection, by the projector, of the pattern of dots within the tracking volume;
initiating capture, by the image capture unit, of at least two images of a portion of the pattern of dots; and
determining three-dimensional positions of dots in the portion of dots from the captured images of the portion of the pattern of dots.

2. The system of claim 1, wherein the operations further comprise determining the three-dimensional position of the one or more markers and the three-dimensional positions of the dots in a same coordinate system.

3. The system of claim 1, wherein the operations further comprise tracking patient anatomy using the three-dimensional positions of the dots.

4. The system of claim 1, wherein determining the three-dimensional positions of the dots comprises using a portion of the dot pattern.

5. The system of claim 4, wherein determining the three-dimensional positions of the dots comprises determining a centroid.

6. The system of claim 1, wherein the operations further comprise matching the dots across the captured images of the portion of the pattern of dots.

7. The system of claim 1, wherein projecting the pattern of dots comprises projecting the pattern of dots in time intervals.

8. The system of claim 7, wherein capturing the at least two images of the portion of the pattern of dots is synchronized with the time intervals.

9. The system of claim 8, wherein the pattern of dots is geometrically changed between subsequent projections.

10. The system of claim 1, wherein the image capture unit comprises multiple cameras.

11. The system of claim 1, wherein the pattern of dots comprises a pseudorandom pattern.

12. The system of claim 1, wherein capturing the images of the one or more markers occurs during a first time period and capturing the images of the portion of the pattern of dots occurs during a second time period, wherein the first time period and the second time period are different.

13. The system of claim 1, wherein the projector is mounted to a housing that contains the image capture unit.

14. The system of claim 1, wherein the projector is positioned remote from a housing that contains the image capture unit.

15. A system comprising:
a projector configured to project a pattern of dots within a tracking volume, wherein a medical instrument having one or more markers is positioned within the tracking volume; and
an image capture unit comprising a memory configured to store instructions and a processor to execute the instructions to perform operations comprising:
initiating capture, by the image capture unit, of at least two images of the medical instrument and the one or more markers;
determining a three-dimensional position of the one or more markers from the captured images of the one or more markers;
initiating projection, by the projector, of the pattern of dots within the tracking volume;
initiating capture, by the image capture unit, of at least two images of a portion of the pattern of dots; and
determining three-dimensional positions of dots in the portion of dots from the captured images of the portion of the pattern of dots.

16. The system of claim 15, wherein the operations further comprise determining the three-dimensional position of the one or more markers and the three-dimensional positions of the dots in a same coordinate system.

17. The system of claim 15, wherein the operations further comprise tracking patient anatomy using the three-dimensional positions of the dots.

18. The system of claim 15, wherein determining the three-dimensional positions of the dots comprises using a portion of the dot pattern.

19. The system of claim 18, wherein determining the three-dimensional positions of the dots comprises determining a centroid.

20. A method comprising:
projecting, by a projector, a pattern of dots within a tracking volume, wherein the tracking volume further comprises a medical instrument having one or more markers and the medical instrument is positioned within the tracking volume;
capturing, by an image capture unit, at least two images of the medical instrument and the one or more markers, wherein the image capture unit is configured to capture imagery of the medical instrument and the one or more markers and configured to capture imagery of the pattern of dots within the tracking volume;
determining, by a computer device, a three-dimensional position of the one or more markers from the captured images of the one or more markers;
projecting, by the projector, of the pattern of dots within the tracking volume;
capturing, by the image capture unit, of at least two images of a portion of the pattern of dots; and
determining three-dimensional positions of dots in the portion of dots from the captured images of the portion of the pattern of dots.
